(19) United States
(12) Patent Application Publication
(10) Pub. No.: US 2023/0165587 A1
(43) Pub. Date: Jun. 1, 2023

(54) EXPANDABLE DEVICES FOR TREATING BODY LUMENS

(71) Applicant: Covidien LP, Mansfield, MA (US)

(72) Inventor: Ramon Carrillo, Santa Ana, CA (US)

(21) Appl. No.: 17/997,327

(22) PCT Filed: Apr. 28, 2021
(86) PCT No.: PCT/US2021/070484

§ 371 Date: Oct. 27, 2022

Related U.S. Application Data
(60) Provisional application No. 63/017,332, filed on Apr. 29, 2020.

(51) Int. Cl. A61B 17/12

(52) U.S. Cl. CPC A61B 17/12113; A61B 17/12172

(57) ABSTRACT

Devices, systems, and methods for treating vascular defects are disclosed herein. One aspect of the present technology, for example, includes an occlusive device comprising a mesh having a low-profile state for intravascular delivery to the aneurysm and a deployed state. The mesh may comprise an expandable cage formed of a plurality of mesh stmts. In some embodiments, the cage is configured to receive an embolic material therein.

### CROSS-REFERENCE TO RELATED APPLICATION(S)

The present application claims the benefit of priority to U.S. Patent Application No. 63/017,332, filed Apr. 29, 2020, which is incorporated by reference herein in its entirety.

### TECHNICAL FIELD

The present technology relates to expandable devices for treating body lumens. Particular embodiments are directed to intrasaccular occlusive devices for treating aneurysms.

### BACKGROUND

Intracranial saccular aneurysms occur in 1% to 2% of the general population and account for approximately 80% to 85% of non-traumatic subarachnoid hemorrhages. Recent studies show a case fatality rate of 8.3% to 66.7% in patients with subarachnoid hemorrhage. Endovascular treatment of intracranial aneurysms emerged in the 1990s with the advent of the Guglielmi detachable coil system (Boston Scientific, Natick, Mass.), which includes packing the aneurysm sac with metal coils to reduce or disrupt the flow of blood into the aneurysm, thereby enabling a local thrombus or clot to form which fills and ultimately closes off the aneurysm. The use of coil embolization to treat aneurysms substantially increased after the publication of favorable clinical data, including evidence that disability or death at the 1-year follow-up occurred in 30.9% of patients treated surgically but only 23.5% in patients treated with coil embolization.[4] Similarly, these trials showed the overall morbidity and mortality at 1 year was 12.6% for surgical clipping and 9.8% for endovascular coiling (amongst patients with no prior history of subarachnoid hemorrhage).

Although coiling has proven to have better outcomes than surgical clipping for both ruptured and unruptured aneurysms, treating complex aneurysms using conventional coiling is challenging. This is especially true for wide-necked aneurysms. Coil segments may protrude from the aneurysm sac through the neck of the aneurysm and into the parent vessel, causing serious complications for the patient. To address this, some treatments include temporarily positioning a balloon within the parent vessel across the neck of the aneurysm to prevent the coils from migrating across the neck during delivery. Alternatively, some treatments include permanently positioning a neck-bridging stent within the parent vessel across the neck of the aneurysm to prevent the coils from migrating across the neck during delivery. While balloon-assisted or stent-assisted coiling for wide-neck aneurysms has shown better occlusion rates and lower recurrence than coiling alone, the recanalization rate of treated large/giant aneurysms can be as high as 18.2%. Moreover, the addition of a balloon or stent and its associated delivery system to the procedure increases the time, cost, and complexity of treatment. Deployment of the stent or balloon during the procedure also greatly increases the risk of an intraprocedural clot forming, and can damage the endothelial lining of the vessel wall. Permanently positioning a stent within the parent vessel increases the chronic risk of clot formation on the stent itself and associated ischemic complications, and thus necessitates the use of dual antiplatelet therapy (“DAPT”). DAPT, in turn, increases the risk and severity of hemorrhagic complications in patients with acutely ruptured aneurysms or other hemorrhagic risks. Thus, neck-bridging stents are not indicated for the treatment of ruptured aneurysms.

The above-noted drawbacks associated with balloon- and stent-assisted coiling techniques influenced the development of intraluminal flow diverting stents, or stent-like structures implanted in the parent vessel across the neck of the aneurysm that redirect blood flow away from the aneurysm, thereby promoting aneurysm thrombosis. Flow diverters have been successfully used for treating wide-neck, giant, fusiform, and blister-like aneurysms. However, because they are positioned in the parent vessel, flow diverters require DAPT to avoid clot formation on the stent itself and ischemic complications. This, in turn, increases the risk and severity of hemorrhagic complications in patients with acutely ruptured aneurysms or other hemorrhagic risks. Thus, flow diverters are not indicated for the treatment of ruptured aneurysms. Flow diverters have also shown limited efficacy in treating bifurcation aneurysms (35-50%).

Endosaccular flow disrupting devices have been gaining momentum over the last decade, generally driven by their potential to provide the intra-aneurysmal flow disruption of coiling with the definitive remodeling at the aneurysm-parent vessel interface achieved by intraluminal flow diverters. Currently existing endosaccular devices are typically mesh devices configured to be deployed completely within the aneurysm sac, with the interstices of the mesh covering the aneurysm neck and reconstructing the aneurysm-parent vessel interface. The implant disrupts the blood flow entering and exiting the aneurysm sac (resulting in stasis and thrombosis) and supports neoendothelial overgrowth without requiring DAPT (unlike endoluminal flow diverters). Thus, endosaccular devices can be used to treat wide-necked aneurysms and ruptured aneurysms. Moreover, because the device is placed completely within the aneurysm sac, the parent and branch vessels are unimpeded and can be accessed for any further retreatment or subsequent deployment of adjunctive devices during treatment.

One existing endosaccular flow disrupting device is the Woven EndoBridge (WEB®; Microvention, Aliso Viejo, Calif.). The WEB device is designed to be placed completely within the aneurysm sac and span the neck where it disrupts local flow. The device is a generally globular, radially symmetrical braid joined at its proximal, centrally located pole to a detachment zone of a delivery wire and is intended to be used as a stand-alone therapy. While the WEB device has had some success in treating classic wide-necked bifurcation aneurysms, its ability to treat a wide range of aneurysm locations, shapes and sizes remains limited. For example, because of its bulky and stiff delivery profile the WEB device is difficult to maneuver around tight turns and thus cannot adequately access the aneurysm sac to treat sidewall aneurysms. Similarly, the larger constrained size of the WEB device requires delivery through a microcatheter having a diameter of at least 0.021 inches, and thus the WEB device cannot access and treat aneurysms at the smaller, more distal intracranial vessels. In addition, because of its globular shape, the WEB device also cannot treat irregularly-shaped aneurysms and is limited to the much-less-common “berry” shaped aneurysms.

Another current endosaccular flow disrupting technology is the Contour Neurovascular System™ (Cerus Endovascular, Fremont, Calif.). The Contour device is constructed from a dual-layer radiopaque shape-memory mesh having a flat, disc-like shape in its fully unconstrained configuration joined at its proximal, centrally located pole to a detachment zone of a delivery wire and is intended to be used as a stand-alone therapy. After deployment, the device assumes a tulip-like configuration conforming to the wall of the lower hemisphere of the aneurysm and across the neck opening. The device is intentionally oversized to the neck and largest measured equatorial diameter of the aneurysm. It can be reloaded and deployed a number of times, permitting an operator to reposition across the neck of the aneurysm. The Contour device is designed to sit across the neck with the marker position below the neck in the parent artery. While the Contour device's construction (joined at its proximal, centrally located pole to the detachment zone of a delivery wire) lends itself to treating bifurcation aneurysms (where the neck is generally normal to/axially aligned with the parent vessel through which the device approaches the aneurysm), its construction does not lend itself to treating side wall aneurysms (where the aneurysm neck is generally parallel to/radially adjacent the parent vessel through which the device approaches the aneurysm). If deployed into a sidewall aneurysm, the delivery catheter will have to approach the aneurysm sac from a shallow angle. Rather than assuming a tulip-like configuration conforming to the wall of the lower hemisphere of the aneurysm and across the neck opening, as when deployed into a bifurcation aneurysm, the device will expand on an angle such that at least the distal edge of the disk will traverse the neck and extend into the parent vessel. This leaves the aneurysm inadequately treated and increases the risk of ischemic complications related to clot formation on the portion of the disk extending into the parent vessel.

The NeQstent™ Aneurysm Bridging Device (Cerus Endovascular, Fremont, Calif.) derives from the Contour device, also having a flat, disc-like shape in its fully unconstrained configuration that is joined at its proximal, centrally located pole to a detachment zone of a delivery wire. In contrast to the Contour device, the NeQstent is intended to be used in conjunction with a separate coiling microcatheter and embolization coils. As such, the NeQstent has approximately 30 to 40% of the number of wires in its double layer mesh construction compared to Contour. This is mainly to allow access through the mesh or between the mesh and aneurysm wall by a coiling microcatheter. Proceeding through or around the mesh is largely dictated by the size and shape of aneurysm and the corresponding device selected. Accordingly, the more the device is oversized to the aneurysm, the more the mesh at the neck of the device is constrained. Once the device and coiling microcatheter are positioned in a preferred position, embolization coils are delivered into the aneurysm until a desired fill is achieved. The microcatheter is then removed and the device is detached from its delivery wire. Like the Contour device, the NeQstent's construction (joined at its proximal, centrally located pole to the detachment zone of a delivery wire) lends itself to treating bifurcation aneurysms but not side wall aneurysms. If deployed into such an aneurysm, rather than assuming a tulip-like configuration conforming to the wall of the lower hemisphere of the aneurysm and across the neck opening, as when deployed into a bifurcation aneurysm, some portion of the disk will traverse the neck and extend into the parent vessel. This leaves the aneurysm neck inadequately protected and increases the risk of ischemic complications related to coil prolapse into the parent vessel or clot formation on the portion of the disk extending into the parent vessel.

Accordingly, there is a need for improved devices and methods for treating aneurysms.

### SUMMARY

The present technology is directed generally to devices, systems, and methods for the treatment of vascular defects, and in particular, to endosaccular occlusive devices for treating ruptured and un-ruptured intracranial wide-neck, bifurcation, and sidewall aneurysms. The occlusive device may comprise an expandable mesh frame. The subject technology is illustrated, for example, according to various aspects described below, including with reference to FIGS. 1A-10.

An occlusive device of the present technology for treating an aneurysm can comprise an expandable cage formed of a plurality of struts, where the cage has a first end region, a second end region, and an intermediate region extending between the first and second end regions. Each of the struts can have a first end portion at the first end of the expandable cage and a second end portion at the second end of the expandable cage. In some embodiments, at least one of the struts comprises a mesh. As compared to traditional stents or baskets formed of a laser cut tube or sheet, the mesh struts of the expandable cage are more flexible and provide. The expandable cage can have a low-profile state for intravascular delivery to the aneurysm and an expanded state in which the expandable cage is configured to be implanted within the aneurysm cavity such that the struts provide an open framework across a neck of the aneurysm.

According to some embodiments, the expandable cage has a substantially prolate spheroid shape in an expanded, unconstrained state. In some embodiments, the expandable cage has a substantially spherical shape in an expanded, unconstrained state. In any of the embodiments disclosed herein, the first end portions of the struts may come together at the first end region of the expandable cage to form a closed, curved first end surface. Additionally or alternatively, the second end portions of the struts may come together at the second end region of the expandable cage to form a closed, curved second end surface.

In some embodiments, the first end portions of the struts are fixed relative to one another and the second end portions of the struts are fixed relative to one another. wherein the at least one strut comprises a braided filament. According to several embodiments the at least one strut comprises an interwoven filament, a helically wound filament, a plurality of braided filaments, a plurality of interwoven filaments, and/or a plurality of helically wound filaments. One, some, or all of the plurality of struts can extend along a longitudinal dimension of the expandable cage. According to several embodiments, the struts are spaced apart about a circumference of the expandable cage along the intermediate region.

In some embodiments, the expandable cage is self-expanding.

According to some embodiments, the mesh is formed of a plurality of filaments. In several of such embodiments, at least some of the filaments are drawn-filled tube (“DFT”) wires.

In some embodiments, the proximal end region of the expandable cage is configured to be detachably coupled to an elongated delivery member. According to several embodiments the first end region comprises a connecting structure. In several of such embodiments, the connecting structure is recessed into a cavity of the expandable cage when the expandable cage is positioned within the aneurysm. In some embodiments, the second end region comprises a connecting structure. In several of such embodiments, the connecting structure is recessed into a cavity of the expandable cage when the expandable cage is positioned within the aneurysm. In any of the embodiments disclosed herein, the expandable cage can comprise one or more struts extending about the circumference of the expandable cage at the intermediate region.

The present technology includes a system for treating an aneurysm comprising the system comprises an embolic material and an expandable cage formed of a plurality of struts, the expandable cage having a first end region, a second end region, and an intermediate region extending between the first and second end regions. In some embodiments, each of the struts have a first end portion at the first end of the expandable cage and a second end portion at the second end of the expandable cage. In some embodiments, at least one of the struts comprises a mesh. In some embodiments, the expandable cage has a low-profile state for intravascular delivery to the aneurysm and an expanded state in which the expandable cage is configured to be implanted within the aneurysm cavity such that the struts provide an open framework configured to receive the embolic material therein. In some embodiments, the embolic material comprises at least one of an embolic coil and a liquid embolic.

In several embodiments, each of the struts comprises a mesh. According to several embodiments, the expandable cage has a substantially prolate spheroid shape in an expanded, unconstrained state. In some embodiments, the expandable cage has a substantially spherical shape in an expanded, unconstrained state. According to some examples, the first end portions of the struts come together at the first end region of the expandable cage to form a closed, curved first end surface. Additionally or alternatively, the second end portions of the struts may come together at the second end region of the expandable cage to form a closed, curved second end surface.

In some embodiments, the first end portions of the struts are fixed relative to one another and the second end portions of the struts are fixed relative to one another. The at least one strut can comprise a braided filament, an interwoven filament, a helically wound filament, a plurality of braided filaments. In some embodiments, one, some, or all of the struts comprise a plurality of interwoven filaments. Additionally or alternatively, one, some, or all of the struts can comprise a plurality of helically wound filaments. One, some, or all of the plurality of struts extends along a longitudinal dimension of the expandable cage.

According to several embodiments, the struts are spaced apart about a circumference of the expandable cage along the intermediate region. The expandable cage is self-expanding. In these and other embodiments, the mesh is formed of a plurality of filaments. In several of such embodiments, at least some of the filaments are DFT wires.

The proximal end region of the expandable cage can be configured to be detachably coupled to an elongated delivery member. In some embodiments, the first end region comprises a connecting structure. In several of such embodiments, the connecting structure can be recessed in a cavity of the expandable cage when the expandable cage is positioned within the aneurysm. In some embodiments the second end region comprises a connecting structure. In several of such embodiments, the connecting structure can be recessed in a cavity of the expandable cage when the expandable cage is positioned within the aneurysm. In any of the embodiments disclosed herein, the expandable cage can comprise a circumferential strut extending about the circumference of the expandable cage at the intermediate region.

The present technology comprises a method of treating an aneurysm. In some embodiments, the method comprises positioning an expandable cage in an aneurysm cavity. The expandable cage can comprise a plurality of struts and have a first end region, a second end region, and an intermediate region extending between the first and second end regions. In some embodiments, each of the struts have a first end portion at the first end of the expandable cage and a second end portion at the second end of the expandable cage. One, some, or all of the struts can comprise a mesh. In some embodiments, the expandable cage has a low-profile state for intravascular delivery to the aneurysm. According to several examples, the method includes positioning a catheter between the struts such that a distal opening of the catheter is disposed within an interior volume of the expandable cage.

The method of Clause 45, further comprising delivering an embolic material to the interior volume of the expandable cage. In some embodiments, the embolic material is at least one of an embolic coil or a liquid embolic.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a side view of an occlusive device configured in accordance with several embodiments of the present technology.

FIG. 1B is an end view of the occlusive device shown in FIG. 1A.

FIG. 2 is a strut configured for use with the occlusive devices of the present technology.

FIG. 3 schematically depicts an occlusive device of the present technology positioned within an aneurysm and with an embolic coil positioned therein.

FIG. 4A is a side view of an occlusive device configured in accordance with several embodiments of the present technology.

FIG. 4B is a side view of an end portion of an occlusive device configured in accordance with several embodiments of the present technology.

FIG. 5 is a side view of an occlusive device configured in accordance with several embodiments of the present technology.

FIG. 6 is a side view of an occlusive device configured in accordance with several embodiments of the present technology.

FIG. 7 is a side view of an occlusive device configured in accordance with several embodiments of the present technology.

FIG. 8 is a side view of an occlusive device configured in accordance with several embodiments of the present technology.

FIG. 9 shows an occlusive device configured in accordance with several embodiments of the present technology.

FIG. 10 schematically depicts the occlusive device of FIG. 9 positioned within a model aneurysm, with a portion of the model removed to better view the occlusive device.

### DETAILED DESCRIPTION

Embolic coils are currently used for embolization of aneurysms but come with several drawbacks. For instance, embolic coils typically do not provide a sufficient surface area for blood to clot, and thus many coils are typically required to fill a single aneurysm, thereby increasing procedural time, cost, and complexity. Additionally, in some cases one or more of the coils may be situated in such a way that creates considerable gaps between adjacent coils, into which blood may freely flow. The addition of extra coils into the aneurysm does not always solve this problem as deploying too many coils into the aneurysm may lead to an undesired rupture. Because the coils are much smaller than the aneurysm cavity, it can be difficult to keep the coils confined to the cavity. As a result, often times one or more of the coils may protrude into the parent vessel with potentially fatal consequences for the patient.

A current approach to treating saccular aneurysms that seeks to avoid the foregoing issues includes covering the neck of the aneurysm with an intravascularly delivered device (also known as a flow diverter). In one variation of this approach, the device is positioned within the parent vessel and thus blocks blood from flowing into the aneurysm cavity from an extrasaccular position. However, because the device is placed within the parent vessel, extrasaccular flow diverters require dual-antiplatement treatment, which comes with increased complications for the patient. Another type of flow diverter is positioned within the aneurysm cavity and thus blocks blood from flowing into the cavity from an intrasaccular position. Examples of commercially available intrasaccular flow diverters are the WEB® SL and WEB® SLS device (Microvention, Aliso Viejo, Calif., USA). Intrasaccular flow diverters, however, are challenging to size, as every aneurysm has a unique shape and a neck width. If the device is too small, for example, it may herniate into the parent vessel with potentially fatal consequences for the patient.

As discussed herein, the occlusive devices of the present technology advantageously address all of the foregoing challenges. The present technology may comprise, for example, an expandable cage configured to be placed within the aneurysm, as opposed to an intravascularly placed stent, which eliminates the need for dual anti-platelet treatment and which lowers procedural complications. The expandable cage can be formed of a plurality of mesh struts, each of which provides an increased surface area for clot formation as opposed to a coil or a solid strut. Moreover, the expandable cage is configured to provide a flexible frame for receiving and supporting one or more embolic coils therein. The mesh struts of the present technology are significantly more flexible than the struts of a traditional stent. For example, the mesh struts of the present technology can flex and bend at any location along their respective longitudinal axes, including at multiple locations simultaneously such that a corresponding strut includes multiple inflection points and/or concavities along its length. Each of the mesh struts herein can also stretch and compress along their respective longitudinal axes, in unison or independently of one another. As a result, the expandable cage of the present technology can adapt and conform to a variety of aneurysm shapes and sizes.

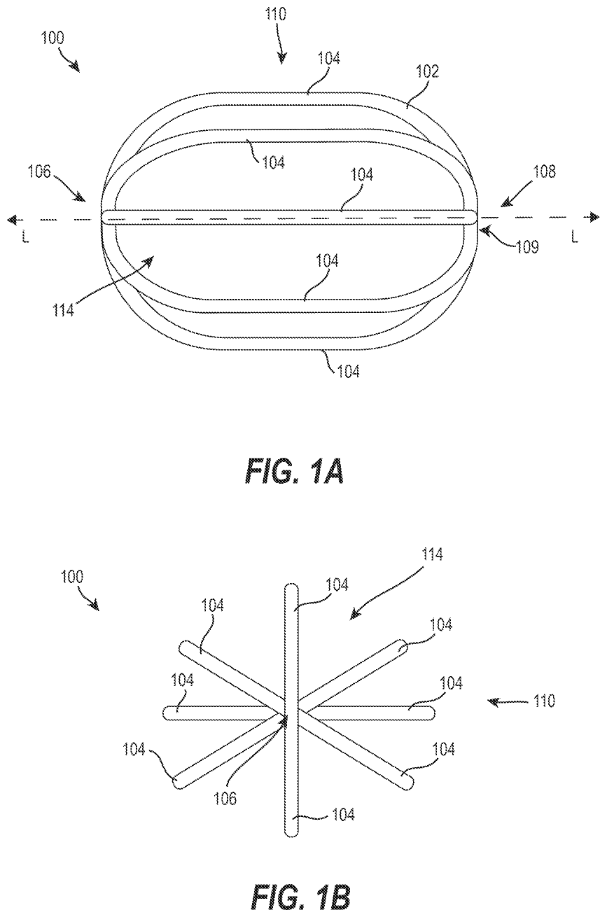

FIGS. 1A and 1B are side and end views, respectively, of an occlusive device 100 (or “device 100”) configured in accordance with several embodiments of the present technology. The occlusive device 100 may comprise a flexible mesh cage 102 configured to be implanted within an aneurysm, such as a cerebral aneurysm. The cage 102 comprises a first end region 106, a second end region 108, and an intermediate region 110 extending between the first and second end regions 106, 108 along a longitudinal dimension L of the cage 102. As shown in FIGS. 1A and 1B, the cage 102 may comprise a plurality of longitudinally-extending mesh struts 104. For ease of illustration, the struts 104 are shown in FIGS. 1A and 1B in a solid (non-porous) form. In an expanded state, the struts 104 may be spaced apart at least along the intermediate region 110 and together define an interior volume 114 of the cage 102. In some embodiments, the interior volume 114 is configured to receive an embolic material therein, such as one or more embolic coils and/or a liquid embolic. The cage 102 thus functions as both a framing structure configured to support and retain an embolic material and as an anchoring structure that self-anchors within the aneurysm. The cage 102 can additionally aid in reducing blood flow into the aneurysm.

The occlusive device 100 and/or cage 102 has a low-profile state (not shown) for intravascular delivery to the aneurysm and a deployed state in which the device 100 is configured to be positioned within the interior cavity of the aneurysm. According to some aspects of the technology, the occlusive device 100 is configured to be advanced through a microcatheter as small as a 0.017-inch microcatheter. When the device 100 is implanted, at least one of the struts 104 is configured to be positioned over at least a portion of the neck of the aneurysm while the subsequently delivered coil(s) fill space within the aneurysm cavity. Positioned across at least a portion of the neck, the cage 102 reduces blood flow entering the sac of the aneurysm, prevents herniation of the coil(s) through the neck and into the parent vessel, and provides a scaffolding that promotes endothelialization across the covered portion of the neck, thus further reducing inflow.

According to several embodiments, for example as shown in FIGS. 1A and 1B, the cage 102 may be biased towards having a prolate spheroid (or watermelon) shape when in an expanded, unconstrained configuration. Despite having this preset shape, the cage 102 may retain enough flexibility such that the cage 102 may deform as necessary to conform to all or a portion of the shape of the aneurysm. One, some, or all of the struts 104 may be generally linear along the intermediate region 110 of the cage 102 and curve towards and/or along the first and/or second end portions 106, 108. In such embodiments, the cage 102 may have a generally constant diameter along the intermediate portion 110. In some embodiments, one, some, or all of the struts 104 may be curved along the intermediate region 110 as well as at the first and/or second end portions 106, 108. In some embodiments, the cage 102 may include a flattened end face 109 at one or both of the end portions 106, 108. In some embodiments, the cage 102 may include a curved end face at one or both of the end portions 106, 108.

In some embodiments, for example as shown in FIG. 1A, the struts 104 may be shaped such that, when the cage 102 is in an expanded configuration, the struts 104 do not radially overlap with one another. In some embodiments, the occlusive device 100 may comprise multiple cages 102 having the same or different shapes. The cages 102 may be arranged end-to-end or may be nested.

Depending on the geometry of the aneurysm to be treated, the cage 102 may have other shapes or configurations and may be formed in a similar manner on molds having other shapes or sizes, such as spherical and non-spherical shapes, cylinders, hemispheres, polyhedrons (e.g., cuboids, tetrahedrons (e.g. pyramids), octahedrons, prisms, etc.), oblate spheroids, plates (e.g., discs, polygonal plates), bowls, non-spherical surfaces of revolution (e.g., toruses, cones, cylinders, or other shapes rotated about a center point or a coplanar axis), and combinations thereof. In its expanded state, the cage 102 may have a size and shape suitable for fitting snugly within a vascular cavity or vesicle (e.g., an aneurysm, or perhaps, a fistula). In some embodiments, the cage 102 may comprise multiple portions of different substantially spherical sizes, which when relaxed and in the expanded configuration nest concentrically, or non-concentrically, with each other within the vascular cavity.

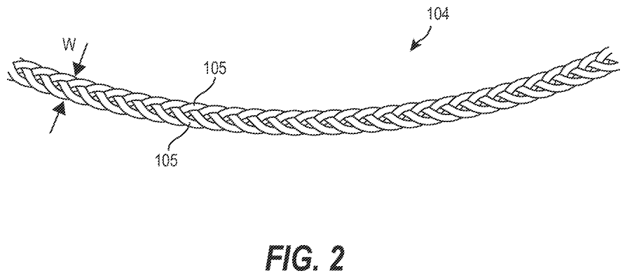

While the cage embodiments represented by FIGS. 1A and 1B are comprised entirely of mesh struts 104, in some embodiments fewer than all of the struts 104 comprise a mesh. According to several embodiments, only select portions of one, some, or all of the struts 104 comprise a mesh. Likewise, the cage 102 may comprise any suitable number of mesh struts. The different struts 104 may have the same or different widths w (see FIG. 2), and a width w of the individual struts 104 may be generally constant or vary along a length of the respective strut 104. One, some, or all of the struts 104 can be formed of the same filament or filaments, which can be braided or interwoven with itself and/or with other filaments. In some embodiments, one, some, or all of the struts 104 are formed of a different filament or filaments, which can be braided or interwoven with itself and/or with other filaments. In some embodiments, the entire cage is made of a single filaments or plurality of filaments.

According to some embodiments, for example as shown in the enlarged view of a portion of one of the struts 104 in FIG. 2, the individual struts 104 may comprise a single strand of one or more filaments 105 that has been helically wound, woven, or braided in a generally linear fashion. The strand may be positioned around an appropriately shaped form or fixture and heat-treated in such a fashion that it will retain its shape after removal from the heating form. For example, the strands may be positioned on or around a fixture having a generally spherical or prolate spheroid shape such that the resulting strut is curved along its length in the expanded state.

In some embodiments, all or a portion of one, some, or all of the struts 104 may be formed of a flattened, tubular braid such that each strut 104 comprises two mesh layers that meet at folds at the side edges of the strut 104. For example, in some embodiments, all or a portion of one, some, or all of the struts 104 may be formed of a tubular braid that has been heat set after being flattened on a flat mandrel such that opposing portions of the tubular sidewall are pressed toward one another along the length of the tubular braid, thereby “flattening” the tubular braid while conforming the braid to planar shape of the mandrel. In some embodiments, one, some, or all of the struts 104 comprise a single layer of mesh.

One, some, or all of the struts 104 can have the same cross-sectional dimension or different cross-sectional dimensions. One, some, or all of the struts 104 can have the same cross-sectional shape or different cross-sectional shapes. Suitable shapes include a rectangle, an square, a circle, an oval, and/or other shapes.

In some embodiments, one, some, or all of the struts 104 have an inner lumen surrounded by a mesh wall that defines the shape of the strut 104.

In those embodiments where one or more of the struts 104 comprise a filament or a plurality of filaments, the mesh strut 104 may be formed of a plurality of wires, at least some of which (e.g., 25% of the wires, 50% of the wires, 80% of the wires, 100% of the wires, etc.) are made of one or more shape memory and/or superelastic materials. Some or all of the wires may have a diameter between about 0.0010 inches and about 0.0012 inches, about 0.0010 inches, about 0.0011 inches, 0.0012 inches (at least prior to etching). In some embodiments, some or all of the wires may be drawn-filled tubes (“DFT”) having a radiopaque core (e.g., platinum) surrounded by a shape memory alloy and/or superelastic alloy (e.g., Nitinol, cobalt chromium, etc.).

All or a portion of the length of some or all of the struts 104 (or components thereof) may have one or more coatings or surface treatments. For example, some or all of the struts 104 may have a lubricious coating or treatment that reduces the delivery force of the device 100 and/or cage 102 as the device 100 is advanced through the delivery catheter. In some embodiments, the coating may be relatively hydrophilic, such as a phosphorocholine compound. Additionally or alternatively, some or all of the struts 104 (or components thereof) may have a coating or treatment (the same as the lubricious coating, or a different coating) that enhances blood compatibility and reduces the thrombogenic surface activity of the braid (e.g., an antithrombogenic coating). In these and other embodiments, at least a portion of the struts 104 (or components thereof) can be made of other suitable materials.

In these and other embodiments, all or a portion of one, some, or all of the struts 104 may be a helically wound coil or any elongated porous structure. In any of the foregoing embodiments, auxiliary fibrous materials may be optionally added to all or a portion of one, some, or all of the struts 104 by weaving, tying, or other suitable permanent attachment methods.

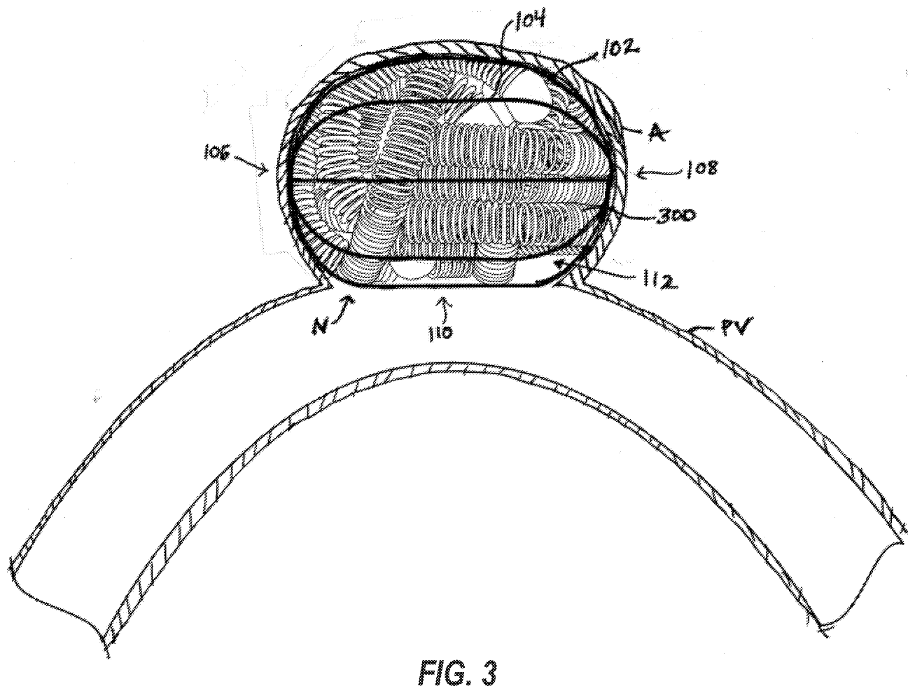

FIG. 3 depicts the cage 102 positioned within an aneurysm cavity A with an embolic coil 300 positioned therein. In use, the device 100 may be intravascularly delivered to the aneurysm cavity in a low-profile configuration within a delivery catheter (e.g., a microcatheter). The distal opening of the delivery catheter may be positioned within the cavity, and the device 100 may be pushed from the distal opening of the delivery catheter into the aneurysm (or the catheter may be withdrawn while holding the device 100 generally stationary). As the device 100 is released from the catheter, the device 100 and/or cage 102 may self-expand into apposition with an inner surface of the aneurysm wall and/or conform to a shape of the aneurysm cavity.

According to some embodiments, an embolic material, such as coil 300, may then be delivered into the interior volume of the cage 102. The embolic material 300 may be delivered through the same catheter that delivered the cage 102, or through a different catheter that has been advanced to the aneurysm A such that a distal portion is proximate or distal of the struts 104 positioned over the neck N. When the physician has been satisfied that the aneurysm is sufficiently occluded, the occlusive device 100 may be detached from the delivery member (such as a pusher member) via one or more detachment mechanisms.

In some cases, the physician may choose to deliver additional coils or embolic material (such as a liquid embolic) to the aneurysm to facilitate delivery, engagement with the aneurysm, or increase of the packing density or fill volume. In these scenarios, the physician may withdraw the pusher member from the delivery catheter and, while maintaining the tip of the delivery catheter within the aneurysm sac (beyond the mesh positioned across the neck), the physician may push the additional embolic material through the delivery catheter and into the aneurysm. The embolic material may comprise one or more liquid embolics, polymers, hydrogels, foams, framing components, and other suitable embolic elements. Any of these embodiments can increase the packing density or fill volume to avoid recanalization of the aneurysm.

Some or all of the cage 102 and/or struts 104 may comprise a radiopaque material. The methods of the present technology may be performed under fluoroscopy such that the radiopaque portions of the device 100 and/or cage 102 may be visualized by the physician to ensure proper neck coverage.

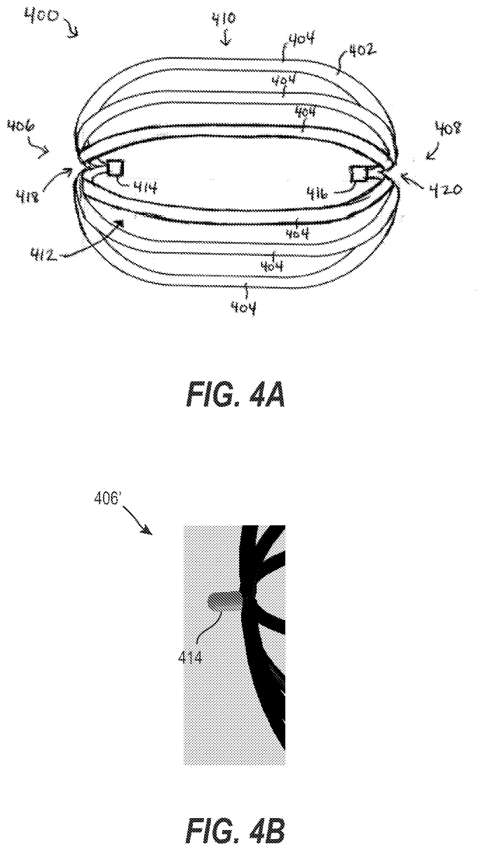

It will be appreciated that one, some, or all of the features and methods described with respect to FIGS. 1A-3 may be incorporated into any of the embodiments disclosed herein, including those depicted in and described with respect to FIGS. 4A-9.

FIG. 4A is a side view of an occlusive device 400 configured in accordance with several embodiments of the present technology. The occlusive device 400 may comprise a flexible mesh cage 402 configured to be implanted within an aneurysm, such as a cerebral aneurysm. The cage 402 comprises a first end region 406, a second end region 408, and an intermediate region 410 extending between the first and second end regions 406, 408 along a longitudinal dimension L of the cage 402. As shown in FIG. 4A, the cage 402 may comprise a plurality of longitudinally-extending mesh struts 404. In an expanded state, the struts 404 may be spaced apart at least along an intermediate region 410 of the cage 402 and together define an interior volume 412 of the cage 402. In some embodiments, the interior volume 412 is configured to receive an embolic material therein, such as one or more embolic coils. The cage 402 thus functions as both a framing structure configured to support and retain an embolic material and as an anchoring structure that self-anchors within the aneurysm. The cage 402 can additionally aid in reducing blood flow into the aneurysm.

As shown in FIG. 4A, in some embodiments the first end portions of the struts 404 may be coupled to one another at a hub 414 that is recessed within the interior cavity 412 of the cage 402. Additionally or alternatively, the second end portions of the struts 404 may be coupled to one another at a hub 414 that is recessed within the interior cavity 412 of the cage 402. As shown in the enlarged view of end portion 406′ in FIG. 4B, in some embodiments the first and/or second end portions of the expandable cage may include a hub 414 oriented away from and/or positioned outside of the internal cavity of the cage 402. In some embodiments, the end portions of the struts 404 and/or the end portions of the individual filaments comprising the struts 404 may be coupled at the first and/or second portion by a coupler 414, such as band. The coupler 414 may comprise a radiopaque material to improve visibility of the device 400 during delivery and after implantation. In some embodiments, the end portions of the struts 404 and/or the end portions of the individual filaments comprising the struts 404 may be coupled at the first and/or second portion (e.g., via crimping, an adhesive, etc.) then welded to form a rounded, atraumatic terminus. Other means for joining the struts and/or individual filaments of the struts are possible. It will be appreciated that one, some, or all of the features described with respect to FIGS. 4A and 4B may be incorporated into any of the embodiments disclosed herein, including those depicted in and described with respect to FIGS. 1A and 1B, 3 and 5-9.

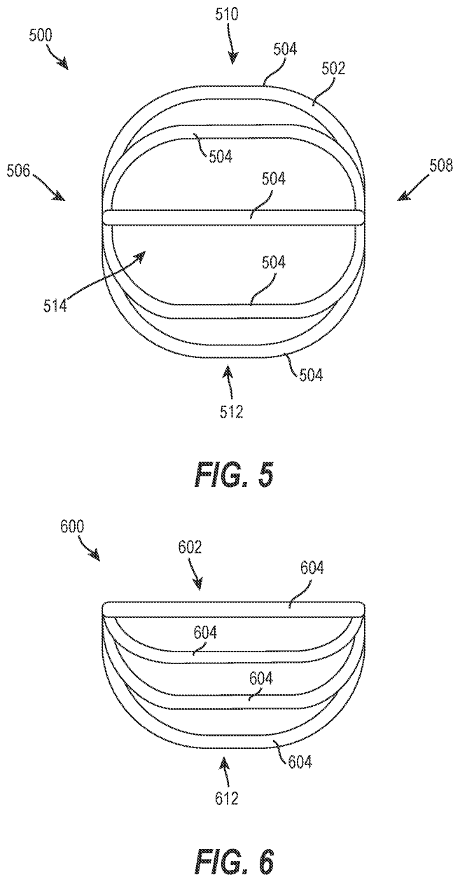

FIG. 5 is a side view of an occlusive device 500 configured in accordance with several embodiments of the present technology. The occlusive device 500 may comprise a flexible mesh cage 502 configured to be implanted within an aneurysm, such as a cerebral aneurysm. The cage 502 comprises a first end region 506, a second end region 508, and an intermediate region 510 extending between the first and second end regions 506, 508 along a longitudinal dimension L of the cage 502. As shown in FIG. 5, the cage 502 may comprise a plurality of longitudinally-extending mesh struts 504. In an expanded state, the struts 504 may be spaced apart at least along an intermediate region 510 of the cage 502 and together define an interior volume 512 of the cage 502. In some embodiments, the interior volume 512 is configured to receive an embolic material therein, such as one or more embolic coils. The cage 502 thus functions as both a framing structure configured to support an embolic material positioned therein, as well as an occlusive element enabled by the increased surface area of the mesh struts 504. In contrast to the watermelon shape of the cage 102 of FIG. 1A, the cage 502 of FIG. 5 is more ball-shaped with flattened end faces 506 and 508. It will be appreciated that any of the embodiments disclosed herein may comprise a flattened end face at the first end portion, the second end portion, or both. Likewise, any of the embodiments disclosed herein may have a rounded or curved end face at the first end portion, the second end portion, or both. It will be appreciated that one, some, or all of the features described with respect to FIG. 5 may be incorporated into any of the embodiments disclosed herein, including those depicted in and described with respect to FIGS. 1A and 1B, 3, 4A and 4B and 6-9.

FIG. 6 is a side view of an occlusive device 600 configured in accordance with several embodiments of the present technology. The occlusive device 600 may comprise a flexible mesh cage 602 configured to be implanted within an aneurysm, such as a cerebral aneurysm. The cage 602 comprises a first end region 606, a second end region 608, and an intermediate region 610 extending between the first and second end regions 606, 608 along a longitudinal dimension L of the cage 602. As shown in FIG. 6, the cage 602 may comprise a plurality of longitudinally-extending mesh struts 604. In an expanded state, the struts 604 may be spaced apart at least along an intermediate region 610 of the cage 602 and together define an interior volume 612 of the cage 602. In some embodiments, the interior volume 612 is configured to receive an embolic material therein, such as one or more embolic coils. The cage 602 thus functions as both a framing structure configured to support an embolic material positioned therein, as well as an occlusive element enabled by the increased surface area of the mesh struts 604. In contrast to the ball-shaped cage 102 of FIG. 1A, the cage 602 of FIG. 6 has a chalice or hollowed, half-spherical shape. It will be appreciated that one, some, or all of the features described with respect to FIG. 6 may be incorporated into any of the embodiments disclosed herein, including those depicted in and described with respect to FIGS. 1A and 1B, 3, 4A and 4B, 5 and 7-9.

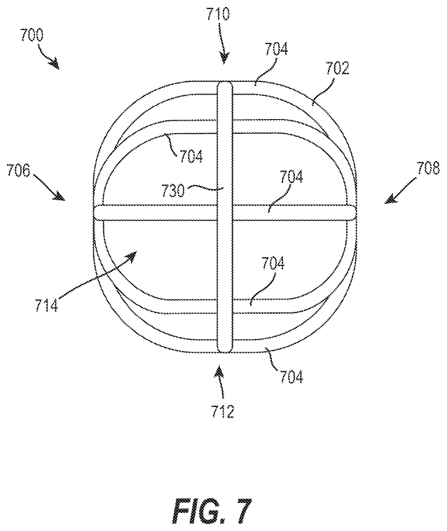

FIG. 7 is a side view of an occlusive device 700 configured in accordance with several embodiments of the present technology. The occlusive device 700 may comprise a flexible mesh cage 702 configured to be implanted within an aneurysm, such as a cerebral aneurysm. The cage 702 comprises a first end region 706, a second end region 708, and an intermediate region 710 extending between the first and second end regions 706, 708 along a longitudinal dimension L of the cage 702. As shown in FIG. 7, the cage 702 may comprise a plurality of longitudinally-extending mesh struts 704. In an expanded state, the struts 704 may be spaced apart at least along an intermediate region 710 of the cage 702 and together define an interior volume 712 of the cage 702. In some embodiments, the interior volume 712 is configured to receive an embolic material therein, such as one or more embolic coils. The cage 702 thus functions as both a framing structure configured to support an embolic material positioned therein, as well as an occlusive element enabled by the increased surface area of the mesh struts 704. As shown in FIG. 7, in some embodiments the occlusive devices and/or cages of the present technology may include one or more circumferentially-extending struts 704. Such struts may comprise a mesh along all or a portion of its respective length, or may be formed of other suitable structures. It will be appreciated that one, some, or all of the features described with respect to FIG. 7 may be incorporated into any of the embodiments disclosed herein, including those depicted in and described with respect to FIGS. 1A and 1B, 3, 4A and 4B, 5, 6, 8 and 9.

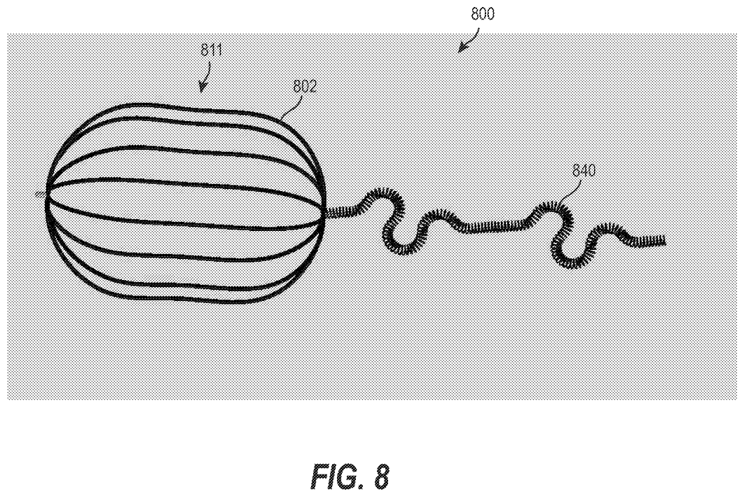

FIG. 8 is a side view of an occlusive device 800 configured in accordance with several embodiments of the present technology. As shown in FIG. 8, in some embodiments the occlusive device 800 includes an embolic coil 840 coupled to an end portion of the cage 802 such that the cage 802 and coil 840 are delivered to the aneurysm together. The coil 840 may have a predetermined shape, and/or may be configured such that, once implanted, the coil 840 is disposed with the internal cavity of the cage 802. Also as shown in FIG. 8, in some embodiments one, some, or all of the struts may have a concavity 811 along the intermediate portion such that the struts extend radially outwardly from the first end portion then radially inwardly at the concavity 811, then radially outwardly again, then inwardly back to the second end portion. In some embodiments, the concavity 811 may face toward an interior region of the cage 802 (as shown in FIG. 8), and in some embodiments the concavity faces radially outwardly, away from the interior region. The cage and/or struts may have multiple concavities along a longitudinal dimension and/or circumferential dimension of the cage. It will be appreciated that one, some, or all of the features described with respect to FIG. 8 may be incorporated into any of the embodiments disclosed herein, including those depicted in and described with respect to FIGS. 1A and 1B, 3, 4A and 4B, 5-7 and 9.

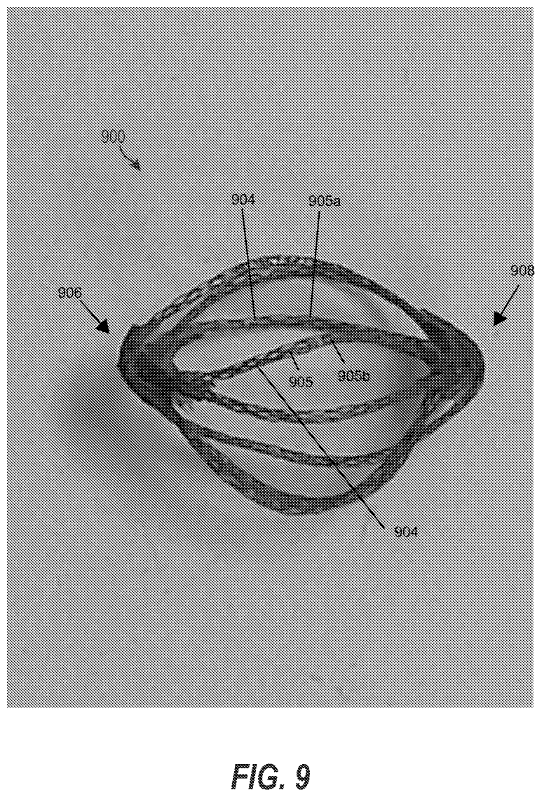

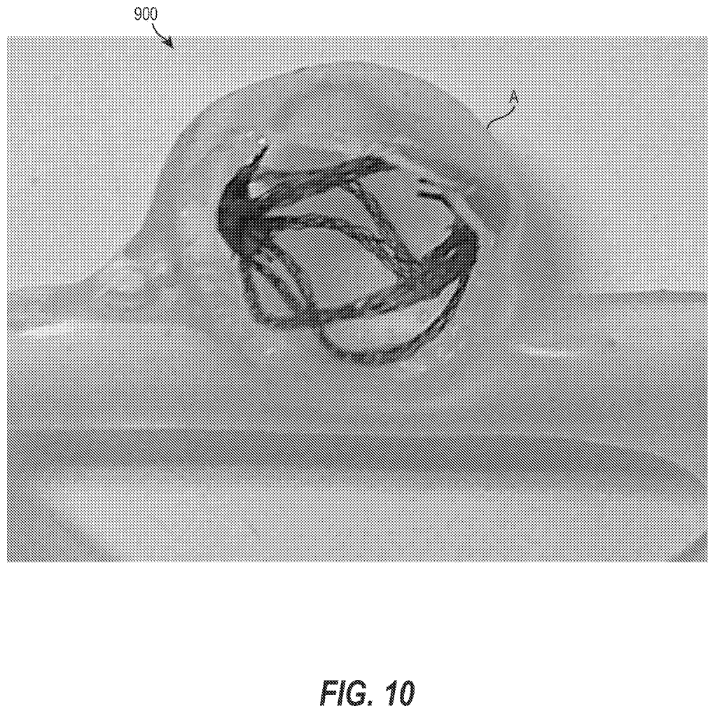

FIG. 9 shows an occlusive device 900 configured in accordance with several embodiments of the present technology, and FIG. 10 schematically depicts the occlusive device 900 of FIG. 9 positioned within a model aneurysm, with a portion of the model removed to better view the occlusive device. As shown in FIGS. 9 and 10, the occlusive device 900 can comprise an expandable cage having a plurality of mesh struts 904 (only a few labeled). The mesh struts 904 can be formed by joining two or more looped elongated meshes such that a first portion 905a of a single looped, elongated mesh 905 can form one of the struts 904 and another portion 905b of the same looped, elongated mesh 905 can form another one of the struts 904. The looped meshes can be joined where they intersect via a coupler, such as an adhesive, a tie, a band, a fused and/or welded joint, etc. In some embodiments, the joined ends comprise the first and second end regions 906, 908 of the expandable cage 902. It will be appreciated that one, some, or all of the features described with respect to FIG. 8 may be incorporated into any of the embodiments disclosed herein, including those depicted in and described with respect to FIGS. 1A and 1B, 3, 4A and 4B and 5-8.

### CONCLUSION

Although many of the embodiments are described above with respect to systems, devices, and methods for treating a cerebral aneurysm, the technology is applicable to other applications and/or other approaches. For example, the occlusive devices, systems, and methods of the present technology can be used to treat any vascular defect and/or fill or partially fill any body cavity or lumen or walls thereof, such as to treat parent vessel occlusion, endovascular aneurysms outside of the brain, arterial-venous malformations, embolism, atrial and ventricular septal defects, patent ductus arteriosus, and patent foramen ovale. Additionally, several other embodiments of the technology can have different states, components, or procedures than those described herein. It will be appreciated that specific elements, substructures, advantages, uses, and/or other features of the embodiments described can be suitably interchanged, substituted or otherwise configured with one another in accordance with additional embodiments of the present technology. A person of ordinary skill in the art, therefore, will accordingly understand that the technology can have other embodiments with additional elements, or the technology can have other embodiments without several of the features shown and described above with reference to FIGS. 1A-10.

As used herein, the terms “generally,” “substantially,” “about,” and similar terms are used as terms of approximation and not as terms of degree, and are intended to account for the inherent variations in measured or calculated values that would be recognized by those of ordinary skill in the art.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An occlusive device for treating an aneurysm, the device comprising:
an expandable cage formed of a plurality of struts, the expandable cage having a first end region, a second end region, and an intermediate region extending between the first and second end regions, wherein each of the struts have a first end portion at the first end of the expandable cage and a second end portion at the second end of the expandable cage, wherein at least one of the struts comprises a mesh,
wherein the expandable cage has a low-profile state for intravascular delivery to the aneurysm and an expanded state in which the expandable cage is configured to be implanted within the aneurysm cavity such that the struts provide an open framework across a neck of the aneurysm.

2. The device of claim 1, wherein each of the struts comprises a mesh.

3. The device of claim 1, wherein the expandable cage has a substantially prolate spheroid shape in an expanded, unconstrained state.

4. The device of claim 1, wherein the expandable cage has a substantially spherical shape in an expanded, unconstrained state.

5. The device of claim 1, wherein the first end portions of the struts come together at the first end region of the expandable cage to form a closed, curved first end surface.

6. The device of claim 1, wherein the second end portions of the struts come together at the second end region of the expandable cage to form a closed, curved second end surface.

7. The device of claim 1, wherein the first end portions of the struts are fixed relative to one another and the second end portions of the struts are fixed relative to one another.

8. The device of claim 1, wherein the at least one strut comprises a braided filament.

9. The device of claim 1, wherein the at least one strut comprises an interwoven filament.

10. The device of claim 1, wherein the at least one strut comprises a helically wound filament.

11. The device of claim 1, wherein the at least one strut comprises a plurality of braided filaments.

12. The device of claim 1, wherein the at least one strut comprises a plurality of interwoven filaments.

13. The device of claim 1, wherein the at least one strut comprises a plurality of helically wound filaments.

14. The device of claim 1, wherein each of the plurality of struts extends along a longitudinal dimension of the expandable cage.

15. The device of claim 1, wherein the struts are spaced apart about a circumference of the expandable cage along the intermediate region.

16. The device of claim 1, wherein the expandable cage is self-expanding.

17. The device of claim 1, wherein the mesh is formed of a plurality of filaments, and wherein at least some of the filaments are drawn-filled tube (“DFT”) wires.

18. The device of claim 1, wherein the proximal end region of the expandable cage is configured to be detachably coupled to an elongated delivery member.

19. The device of claim 1, wherein the first end region comprises a connecting structure, the connecting structure being recessed in a cavity of the expandable cage when the expandable cage is positioned within the aneurysm.

20. The device of claim 1, wherein the second end region comprises a connecting structure, the connecting structure being recessed in a cavity of the expandable cage when the expandable cage is positioned within the aneurysm.

21. The device of claim 1, further comprising a circumferential strut extending about the circumference of the expandable cage at the intermediate region.

22-67. (canceled)
